(19) United States
(12) Patent Application Publication
(10) Pub. No.: US 2021/0401733 A1
(43) Pub. Date: Dec. 30, 2021

(54) SKIN CRÈME

(71) Applicant: Bruce Douglas Sheffler, West Frankfort, IL (US)

(72) Inventor: Bruce Douglas Sheffler, West Frankfort, IL (US)

(21) Appl. No.: 16/917,023

(22) Filed: Jun. 30, 2020

(51) Int. Cl. A61K 9/00; A61K 9/06; A61Q 19/08; A61K 47/20; A61K 47/10; A61K 47/44; A61K 47/46; A61K 8/67; A61K 47/22; A61K 47/14; A61K 8/37; A61K 31/047; A61K 8/36; A61K 47/12; A61K 8/55; A61K 47/24; A61K 8/11; A61K 9/48; A61K 8/73; A61K 9/107; A61K 8/06; A61K 36/28; A61K 8/46; A61K 8/34; A61K 8/92; A61K 8/9794; A61K 8/9728

(52) U.S. Cl. CPC A61K 9/0014; A61K 8/9728; A61Q 19/08; A61K 47/20; A61K 47/10; A61K 47/44; A61K 47/46; A61K 8/671; A61K 8/676; A61K 47/22; A61K 8/678; A61K 47/14; A61K 8/37; A61K 31/047; A61K 8/361; A61K 47/12; A61K 8/553; A61K 47/24; A61K 8/11; A61K 9/4816; A61K 8/733; A61K 9/1075; A61K 8/068; A61K 36/28; A61K 8/46; A61K 8/345; A61K 8/922; A61K 8/9794; A61K 9/06

(57) ABSTRACT

A skin crème with CBD variants. The skin crème is a formulation to relieve joint and muscle pain, pain from arthritis. The skin crème also works to repair skin and reduce wrinkles, as the CBD nourishes the skin and has been shown to be more effective in skin repair and wrinkles reduction. There are three embodiments that include different forms of CBD. The first embodiment is a skin crème with CBD oil. The second embodiment is a skin crème with water soluble CBD. The third embodiment is a skin crème with Nano CBD.

### BACKGROUND

The present invention generally relates to a crème for skin. More specifically, the present invention relates to a crème for skin that includes the use of CBD variants.

CBD is an abbreviation for cannabidiol extract. CBD is just one of more than 80 cannabinoid variations extracted from the cannabis plant. Unlike other cannabinoids, CBD bears no psychoactive effects. It is rich in vitamins and has been found to have numerous positive effects on our bodies. CBD is known to be the second most prevalent cannabinoid in the cannabis plant with the reigning one being THC. Plants that usually are cultivated for the extraction of CBD are known as high-CBD, low-THC hemp plants. On the contrary, plants used for medical marijuana are referred to as high-THC plants. As is, hemp is known to contain very minimal traces of THC. It is for this reason that it is mainly used in the textile industry among many other sectors.

Just like many other cannabinoids, CBD tends to interact with our bodies in numerous ways. It is known to interact with a component referred to as the endocannabinoid system, which is a unique set of brain receptors which deal with cannabinoids alone. The human ECS is known to have an impact on pain sensations, appetite, mood, and memory. With the existence of ECS, it is evident that our brains can handle cannabinoids. In fact, our bodies tend to produce cannabinoids which are meant to communicate with the ECS receptors. A majority of people today do not include cannabinoids in their diet, and as such, ECS is not well maintained. Although the internally produce cannabinoids are beneficial, their impact is not the same as that of the natural, plant-based cannabinoids.

There are numerous ways in which you can introduce CBD to your body with the most popular one being through CBD oil. CBD oil is entirely obtained from the stalk of the plant. Though seed extracts do not contain CBD, seed extracts can be added to the oil to boost the oil's nutritional value. The CBD can be used to fight acne, fight Post Traumatic Stress Disorder, treatment of Schizophrenia, and mitigate cigarette cravings. CBD is also known to provide pain relief, reduce the risk of diabetes, mitigate the symptoms of cancer, improve cardiovascular health, and fight nausea.

It is an object of the present invention to provide a crème with CBD to be applied to the skin.

### SUMMARY

A skin crème with CBD variants. The skin crème is a formulation to relieve joint and muscle pain, pain from arthritis. The skin crème also works to repair skin and reduce wrinkles, as the CBD nourishes the skin and has been shown to be more effective in skin repair and wrinkles reduction. There are three embodiments that include different forms of CBD. The first embodiment is a skin crème with CBD oil. The second embodiment is a skin crème with water soluble CBD. The third embodiment is a skin crème with Nano CBD.

### DETAILED DESCRIPTION

The present invention is skin crème with CBD variants. The skin crème is a formulation to relieve joint and muscle pain, pain from arthritis. The skin crème also works to repair skin and reduce wrinkles, as the CBD nourishes the skin and has been shown to be more effective in skin repair and wrinkles reduction. There are three embodiments that include different forms of CBD.

The first embodiment is a skin crème with CBD oil. The CBD oil variant includes Triple UV Treated Deionized Water (Aqua) 15.15%; Dimethyl Sulfone 16% (MSM—Organic Sulfur); Organic Vegetable Glycerin 5%; Organic Prunus armeniaca (Apricot Kernel) Oil 0.1%, Organic Prunus amygdalus (Sweet Almond) Oil 0.1%; Organic Aloe Barbadensis Extract 200:1 5%; Usnea longissima (wild crafted) 0.1%; Organic Calendula officinalis Flower Extract 0.1%; Organic Vitamin A (Palmitate) 0.1%; Organic Vitamins C (Citrate) 1%; Organic Vitamin E (Acetate) 0.25%; Grapefruit Seed Extract Citricidal 0.1%; 100% pure essential oil of Melaleuca alternifolia 0.1%; Fatty Acid Esters derived from coconut pulp used as an emulsifier 50%; Pure 100% CBD 0.83% and MCT Oil 6.7%. Where the Pure 100% CBD 0.83% and MCT Oil 6.7% forms the CBD oil.

The second embodiment is a skin crème with water soluble CBD. The water soluble CBD variant includes Triple UV Treated Deionized Water (Aqua) 17.7%; Dimethyl Sulfone 16% (MSM—Organic Sulfur); Organic Vegetable Glycerin 5%; Organic Prunus armeniaca (Apricot Kernel) Oil 0.1%; Organic Prunus amygdalus (Sweet Almond) Oil 0.1%; Organic Aloe Barbadensis Extract 200:1 5%; Usnea longissima (wild crafted) 0.1%; Organic Calendula officinalis Flower Extract 0.1%; , Organic Vitamin A (Palmitate) 0.1%; Organic Vitamins C (Citrate) 1%; Organic Vitamin E (Acetate) 0.25%; Grapefruit Seed Extract Citricidal 0.1%; 100% pure essential oil of Melaleuca alternifolia 0.1%; Fatty Acid Esters derived from coconut pulp used as an emulsifier 50%; Pure 100% CBD 0.83%; Lecithin 2.26%; Phospholipids 1.51% (These are used to emulsify the Mixture); and Encapsulant sodium alginate 0.38%. Where the Pure 100% CBD 0.83%; and Lecithin 2.26% Phospholipids 1.51% and Encapsulant sodium alginate 0.38% forms the water soluble CBD.

CBD obtained from the plant is initially in an oil form. Our bodies are mostly water, whereby water and oil do not mix well. Water Soluble CBD should more accurately described as Water Compatible CBD. The CBD is mixed with a blend of food-grade (GRAS) carrier oils, emulsifiers, and preservatives, all derived from natural sources. It is then mixed under high frequency until the oil blends with the emulsifiers, oils and preservatives.

Liposomes are spherical structures with diameters from about 50 to 500 nm formed by one or more concentric phospholipid bilayers with an aqueous phase inside and in-between the bilayers. Liposomes can entrap water-soluble (hydrophilic) active ingredients in their internal water compartment and water-insoluble (hydrophobic) active ingredients in their bilayer membrane. The latter property makes it possible to use liposomes to formulate water-compatible cannabis extracts. This, however, involves complex preparation procedures and, as with microemulsions, very high surfactant (lecithin phospholipids) concentrations. In addition, it is difficult to produce liposomal formulations using natural cannabis extracts, requiring the use of synthetic or isolated cannabinoids instead. A typical liposome precursor formulation includes an active ingredient (e.g., synthetic cannabinoids) 18.90%, surfactants (e.g., lecithin phospholipids) 75.60% and an encapsulant (e.g., sodium alginate) 5.50%. The CBD oil is encapsulated within these compounds which are hydrophilic, meaning that they are attracted to water. These agents combined with the reduced droplet size of the CBD oil contained within to make the mixture water soluble or water compatible and increase the bio-availability of the oil contained within the droplets. Typically CBD characterized as

Water Soluble has a nano particle size of greater than 150. Bio-availability is defined as: “the degree and rate of which a substance (such as a drug) is absorbed into a living system or is made available at the site of interaction.”

To make CBD water soluble, the cannabidiol molecule (or oil containing the molecule) must be emulsified by creating a micelle. A micelle is a sphere of phospholipids (like lecithin) that creates a highly bioavailable lipophilic core (a tiny oil droplet containing CBD) that is separated from the bulk water. Surfactant molecules can be used to stabilize the emulsion, preventing micelle rupture and the undesirable oil-water separation.

The CBD oil is then sonicated (vigorously vibrated) in the water solution in small batches to ensure homogeneous emulsification. The lecithin forms the micelle's shell, while the quillaja saponin acts as a surfactant increasing the gap between the lecithin and the surrounding water molecules once water is added. Potassium sorbate is added to prevent bacteria or mold growth, ensuring a safe, aqueous product that can be added to any food or drink with minimal flavor.

The third embodiment is a skin crème with Nano CBD. The Nano CBD variant includes Triple UV Treated Deionized Water (Aqua) 18%; Dimethyl Sulfone 16% (MSM—Organic Sulfur); Organic Vegetable Glycerin 5%; Organic Prunus armeniaca (Apricot Kernel) Oil 0.1%; Organic Prunus amygdalus (Sweet Almond) Oil 0.1%; Organic Aloe Barbadensis Extract 200:1 5%; Usnea longissima (wild crafted) 0.1%; Organic Calendula officinalis Flower Extract 0.1%; Organic Vitamin A (Palmitate) 0.1%; Organic Vitamins C (Citrate) 1%; Organic Vitamin E (Acetate) 0.25%; Grapefruit Seed Extract Citricidal 0.1%; 100% pure essential oil of Melaleuca alternifolia 0.1%; Fatty Acid Esters derived from coconut pulp used as an emulsifier 50%; Pure 100% CBD 0.83%; and Sufficants (to emulsify the CBD and make it hydrophilic and remain in the nano state) 3.85%. Where the Pure 100% CBD 0.83%; and Sufficants 3.85% forms the Nano CBD.

Nano CBD takes Water Soluble CBD a step further. The process is the same except that different surfactants are used that tend to be more expensive and the droplet size of the oil is driven down to the 30 to 100 nm range. Nanoemulsions are emulsions with narrow droplet size distributions centered below approximately 250 nm. Nanoemulsion-based formulations in which all droplets are smaller than 100 nm are optically translucent, achieving progressively higher degree of clarity as the droplet sizes are diminished. These formulations have several attractive properties, including low viscosity, high interfacial surface area and long-term kinetic stability. Nanoemulsions are made using significantly (about 10 times) lower surfactant amounts than microemulsions or liposomes. They are completely water-compatible and can be easily mixed into water or any beverage. A typical nanoemulsion concentrate (with some water, but before the addition to a formulation includes an active ingredient (e.g., cannabis oil extract) 5.40%, Carrier oil (e.g., olive oil, mct oil) 7.20%, Surfactant (e.g., Quillaja saponin) 2.00%, Water 85.4%. The reason that cannabis oil nanoemulsions require much lower surfactant concentrations than the alternatives is that the driving force for their formation is mainly mechanical instead of chemical. Nano emulsions can be produced by utilizing ultrasonic cavitation-derived high shear forces, able to break the oil droplets down to nano meter sizes. A Nano particle is one-billionth of a meter, which is one-millionth of a millimeter. The smaller size of the nano CBD particles makes the Nano many times more bio-available than the water soluble CBD.

Surfactants used in Nano CBD include Polysorbate 80 (also known as Alkest TW 80), Scattics, Canarcel, Poegasorb 80, Montanox 80, and Tween 80. Ultrasonic cavitation is a very effective emulsification method to prepare superior emulsions in the nano range. The sonication of emulsions with turbidities (micro-emulsions, approx. 150 to 100 nm) makes them translucent or clear and transparent, because it reduces the CBD droplet size to diminutive droplets in the range of approx. l0nm. This increases the emulsion stability significantly. Ultrasonically produced emulsions are often self-stable without addition of an emulsifier or surfactant. For cannabis oil, nano emulsification improves cannabinoids absorption (bioavailability) and produces a more profound effect. Therefore lower cannabis product doses can give you the same effects.

Arnica is an herb that grows mainly in Siberia and central Europe, as well as temperate climates in North America. The flowers of the plant are used in medicine.

Arnica is most commonly used for pain caused by osteoarthritis, sore throat, surgery, and other conditions. Arnica is also used for bleeding, bruising, swelling after surgery, and other conditions, but there is no good scientific evidence to support these uses. Arnica can also be unsafe when taken by mouth. In foods, arnica is a flavor ingredient in beverages, frozen dairy desserts, candy, baked goods, gelatins, and puddings. In manufacturing, arnica is used in hair tonics and anti-dandruff preparations. The oil is used in perfumes and cosmetics. The active chemicals in arnica may reduce swelling, decrease pain, and act as antibiotics. Early research shows that using an arnica gel product (A. Vogel Arnica Gel, Bioforce AG) twice daily for 3 weeks reduces pain and stiffness and improves function in people with osteoarthritis in the hand or knee. Other research shows that using the same gel works as well as the painkiller ibuprofen in reducing pain and improving function in the hands.

Methylsulfonylmethane (MSM) is a naturally occurring organosulfur compound utilized as a complementary and alternative medicine (CAM) under a variety of names including dimethyl sulfone, methyl sulfone, sulfonylbismethane, organic sulfur, or crystalline dimethyl sulfoxide. MSM is a member of the methyl-S-methane compounds within the Earth's sulfur cycle. Natural synthesis of MSM begins with the uptake of sulfate to produce dimethylsulfoniopropionate (DMSP) by algae, phytoplankton, and other marine microorganisms. DMSP is either cleaved to form dimethyl sulfide (DMS) or undergoes demethiolation resulting in methanethiol, which can then be converted to DMS. Approximately 1%-2% of the DMS produced in the oceans is aerosolized. Atmospheric DMS is oxidized by ozone, UV irradiation, nitrate (NO3), or hydroxyl radical (OH) to form DMSO or sulfur dioxide. Atmospheric levels of DMSO and MSM appear to be dependent upon the season with a maxima in the spring/summer and minima in the winter, possibly due to DMS production and volatility being temperature dependent. Oxidized DMS products like sulfur dioxide contribute to increased condensation and cloud formation, thus providing a vehicle for DMSO to return to Earth dissolved in precipitation where it can undergo disproportionation to either DMS or MSM. Once absorbed into the soil, DMSO and MSM will be taken up by plants or utilized by mutualistic soil bacterium such as the bioremediative additive, Pseudomonas putida, in order to improve soil conditions. MSM is broadly expressed in a number of fruit, vegetable, and grain crops, though the extent of MSM bioaccumulation is dependent upon the plant. At this point, MSM and the other sulfur sources are consumed as a plant product and excreted, released as a by-product of plant respiration in the form of sulfide, or eventually decompose as the plant dies. The non-aerosolized sulfur sources

1. A skin creme, comprising: Triple UV Treated Deionized Water (Aqua); Dimethyl Sulfone (MSM—Organic Sulfur); Organic Vegetable Glycerin; Organic Prunus armeniaca (Apricot Kernel) Oil; Organic Prunus amygdalus (Sweet Almond) Oil; Organic Aloe Barbadensis Extract 200:1; Usnea longissima (wild crafted); Organic Calendula Officinalis Flower Extract; Organic Vitamin A (Palmitate); Organic Vitamins C (Citrate); Organic Vitamin E (Acetate); Grapefruit Seed Extract Citricidal; 100% pure essential oil of Melaleuca alternifolia; Fatty Acid Esters derived from coconut pulp used as an emulsifier; Pure 100% CBD and MCT Oil.

2. The skin creme of claim 1, wherein said Triple UV Treated Deionized Water (Aqua) is 15.15%; said Dimethyl Sulfone (MSM—Organic Sulfur) is 16%; said Organic Vegetable Glycerin is 5%; said Organic Prunus armeniaca (Apricot Kernel) Oil is 0.1%, said Organic Prunus amygdalus (Sweet Almond) Oil is 0.1%; said Organic Aloe Barbadensis Extract 200:1 is 5%; said Usnea longissima (wild crafted) is 0.1%; Organic Calendula officinalis Flower Extract is 0.1%; said Organic Vitamin A (Palmitate) is 0.1%; said Organic Vitamins C (Citrate) is 1%; said Organic Vitamin E (Acetate) is 0.25%; said Grapefruit Seed Extract Citricidal is 0.1%; said 100% pure essential oil of Melaleuca alternifolia is 0.1%; said Fatty Acid Esters derived from coconut pulp used as an emulsifier is 50%; Pure 100% CBD oil is 0.83% and said MCT Oil is 6.7%.

3. A skin crème, comprising: Triple UV Treated Deionized Water (Aqua); Dimethyl Sulfone (MSM—Organic Sulfur); Organic Vegetable Glycerin; Organic Prunus amygdalus (Sweet Almond) Oil; Organic Aloe Barbadensis Extract 200:1; Usnea longissima (wild crafted); Organic Calendula officinalis Flower Extract;
Organic Vitamin A (Palmitate); Organic Vitamins C (Citrate); Organic Vitamin E (Acetate); Grapefruit Seed Extract Citricidal; 100% pure essential oil of Melaleuca alternifolia; Fatty Acid Esters derived from coconut pulp used as an emulsifier; Pure 100% CBD; Lecithin; Phospholipids for emulsifying the Mixture; and Encapsulant sodium alginate.

4. The skin creme of claim 3, wherein said Deionized Water (Aqua) is 17.7%; said Dimethyl Sulfone is 16%; said Organic Vegetable Glycerin is 5%; said Organic Prunus armeniaca Oil is 0.1%; said Organic Prunus amygdalus Oil is 0.1%; said Organic Aloe Barbadensis Extract 200:1 is 5%; said Usnea longissima (wild crafted) is 0.1%; said Organic Calendula officinalis Flower Extract is 0.1%; said Organic Vitamin A is 0.1%; said Organic Vitamins C is 1%; said Organic Vitamin E is 0.25%; said Grapefruit Seed Extract Citricidal is 0.1%; said 100% pure essential oil of Melaleuca alternifolia is 0.1%; said Fatty Acid Esters is 50%; said Pure 100% CBD is 0.83%; said Lecithin is 2.26%; said Phospholipids is 1.51%; and said Encapsulant sodium alginate is 0.38%.

5. A skin creme, comprising: Triple UV Treated Deionized Water (Aqua); Dimethyl Sulfone (MSM—Organic Sulfur); Organic Vegetable Glycerin; Organic Prunus armeniaca (Apricot Kernel) Oil; Organic Prunus amygdalus (Sweet Almond) Oil; Organic Aloe Barbadensis Extract 200:1; Usnea longissima (wild crafted); Organic Calendula officinalis Flower Extract; Organic Vitamin A (Palmitate); Organic Vitamins C (Citrate); Organic Vitamin E (Acetate); Grapefruit Seed Extract Citricidal; 100% pure essential oil of Melaleuca alternifolia; Fatty Acid Esters derived from coconut pulp used as an emulsifier; Pure 100% CBD; and Sufficants to emulsify the CBD and make it hydrophilic and remain in the nano state.

6. The skin creme of claim 5, wherein said Triple UV Treated Deionized Water is 18%; said Dimethyl Sulfone is 16%; said Organic Vegetable Glycerin is 5%; said Organic Prunus armeniaca Oil is 0.1%; said Organic Prunus amygdalus Oil is 0.1%; said Organic Aloe Barbadensis Extract 200:1 is 5%; said Usnea longissima is 0.1%; said Organic Calendula officinalis Flower Extract is 0.1%; said Organic Vitamin A is 0.1%; said Organic Vitamins C is 1%; said Organic Vitamin E is 0.25%; said Grapefruit Seed Extract Citricidal is 0.1%; said 100% pure essential oil of Melaleuca alternifolia is 0.1%; said Fatty Acid Esters is 50%; said Pure 100% CBD is 0.83%; and said Sufficants is 3.85%.
